(19) United States
(12) Patent Application Publication
(10) Pub. No.: US 2025/0288258 A1
(43) Pub. Date: Sep. 18, 2025

(54) Auto-Dial 911 Mayday Bracelet

(71) Applicant: Keith Alan Roberts, Melbourne, FL (US)

(72) Inventor: Keith Alan Roberts, Melbourne, FL (US)

(21) Appl. No.: 18/901,947

(22) Filed: Sep. 30, 2024

(51) Int. Cl. A61B 5/00; A44C 5/00; A61B 5/024; G16H 10/60; G16H 40/67; H04W 4/90

(52) U.S. Cl. CPC A61B 5/747; A44C 5/0015; A61B 5/02438; A61B 5/4845; A61B 5/681; A61B 5/7264; A61B 5/7405; A61B 5/746; G16H 10/60; G16H 40/67; H04W 4/90

(57) ABSTRACT

Wherein a life-threatening low heart rate is detected, the Mayday Bracelet becomes a life saving method for an unconscious overdosed person whose life is rapidly slipping away, to save their own life, to automatically dispatch a pre-recorded mayday message to a 911 operator, revealing personal information, location, GPS coordinates and requesting sending paramedics, a piercing alarm beeps. Wherein the device is an automated, electronic, low heart rate detecting alarm. A reversible bracelet wearable on either the left or right wrist, contacting the radial arteries, a temperature sensor helps to prevent a false alarm, a real time GPS coordinates locator, longitude and latitude will illuminate on a display screen, wherein an audio recorder a user will pre-record a 911 mayday message, to automatically be dispatched to 911 operator, wherein a life-threatening low heart rate alarm is activated, simultaneously remotely activating a detached GPS beacon with a siren.

### BACKGROUND OF THE INVENTION

Opioids, and especially fentanyl overdose deaths, is an out-of-control epidemic. Wherein the U.S. is being saturated with fentanyl. Wherein last year in 2023 the U.S. recorded over 100,000 opioid overdose deaths, wherein over 60,000 people have been revived with Narcan. Wherein someone who is using opioids is at risk of receiving a lethal dose of fentanyl and death.

Wherein said Narcan is an opioid overdose reversal agent administered intranasally. Wherein said Narcan works great if a responder is around in time to administer said Narcan. Wherein mostly everyone who has died from said opioid overdose did not receive said Narcan.

Wherein an unconscious overdosed person who does not receive said Narcan, occurs if no one is aware of said unconscious overdosed person's dire straits, someone who is home alone, others in a household unaware a roommate is slipping away, or someone homeless. Whereby a Mayday Bracelet wherein detecting a life-threatening low heart rate, a piercing alarm beeps, wherein automatically dispatching a pre-recorded mayday message to a 911 operator, wherein remotely activating a detached GPS beacon with a high decibel siren. Therefore said unconscious overdosed person can be located.

Wherein said Mayday Bracelet with its piercing alarm will alert the attention of loved ones, caretakers, that an in house hospice care person is unconscious and passing away, wherein a chance to say goodbye before it's too late. Wherein parents can tell their kids if they are going to the bars, or plan to spend the night out to please wear said Mayday Bracelet. Wherein said piercing alarm will alert someone in a house that a roommate is unconscious, overdosed and in need of Narcan administered. Wherein someone who is knowingly using opioids, whereby wearing said Mayday Bracelet they are never alone. Safety and peace of mind, early warnings for all.

### SUMMARY OF THE INVENTION

Keeping up with the changing of times, wherein to help combat opioid and especially fentanyl overdose deaths. Wherein said Mayday Bracelet is a last line of defense, a precautionary preemptive method for an unconscious overdosed person to save their own life.

Wherein heartbeat sensors relay data to a microprocessor chip, wherein said heartbeats are converted into countable seconds, wherein a stopwatch second counting alarm is set for one heartbeat every two seconds. Two heartbeats one second apart is 60 BPM two heartbeats two second apart being half, 30 BPM is the life-threatening low heart rate alarm, wherein an overdosed person with a heart rate of 30 BPM is someone unconscious whose life is rapidly slipping away, and will not awake without said Narcan administered.

Wherein said stopwatch second counting low heart rate alarm can be adjusted to 40 BPM. A temperature sensor helps to prevent a false alarm, wherein a push of a button a user can illuminate said GPS coordinates longitude and latitude on a display screen, wherein a push of a button said user can record a mayday message, wherein said pre-recorded mayday message will automatically be dispatched to a 911 operator wherein a life-threatening low heart rate alarm is activated.

Wherein said 911 operator will not receive a silent call, wherein said pre-recorded mayday message will play and repeat in a loop, wherein said 911 operator can call back said Mayday Bracelet to check for a response, wherein a push of a button said user can manually call a 911 operator and communicate via a microphone.

Wherein a direction label indicates the correct inversion of said Mayday Bracelet, whereas heartbeat sensors will contact a radial artery, whether it's worn on the left or right wrist. Wherein a direction label suggests a pre-recorded mayday message to recite. Wherein a LED light on said display screen will flash to a heartbeat rhythm.

### BRIEF DESCRIPTION OF THE DRAWINGS

FIG. 1 is a perspective view; the preferred embodiment is a Mayday Bracelet illustrating left wrist inversion and inner features.

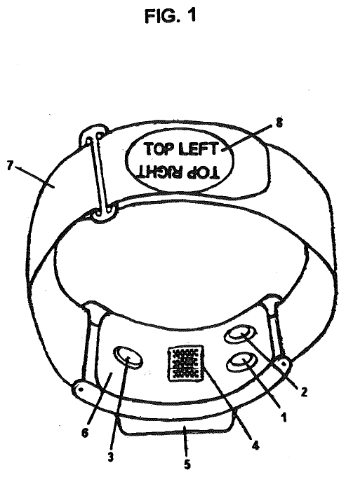

FIG. 2 is a perspective view; the preferred embodiment is a Mayday Bracelet illustrating right wrist inversion and inner features.

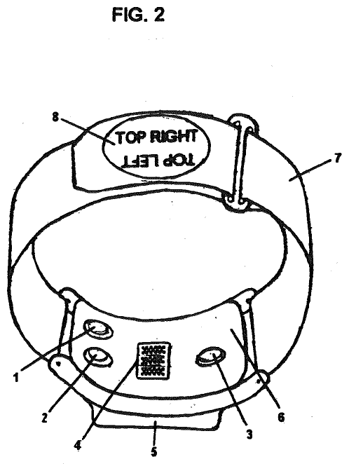

FIG. 3 is a perspective view; the embodiments are a left and right wrist wearing a reversible Mayday Bracelet, illustrating heartbeat sensors contacting radial arteries.

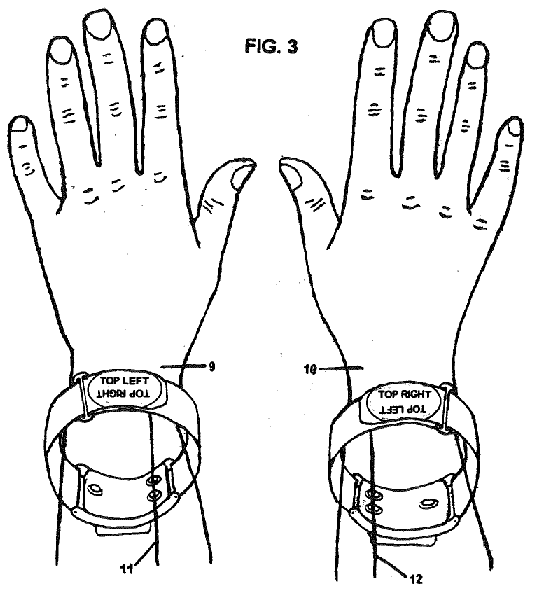

FIG. 4 is a perspective view; the preferred embodiment is a Mayday Bracelet, illustrating outer features.

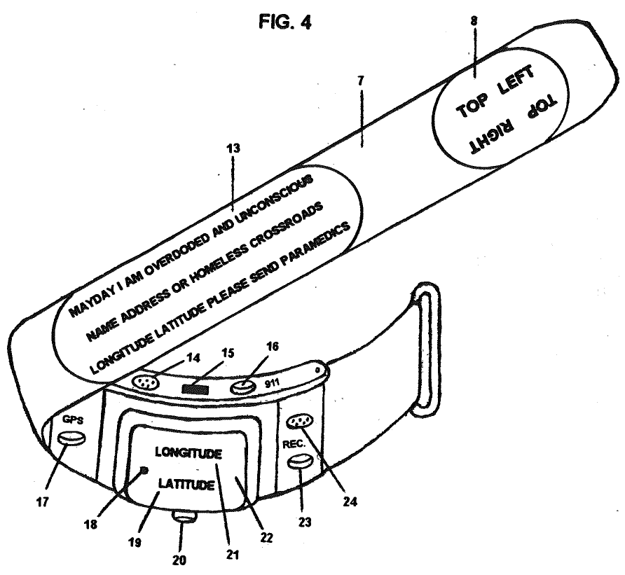

FIG. 5 is a perspective view; the preferred embodiment is a GPS locating beacon, siren and outer features.

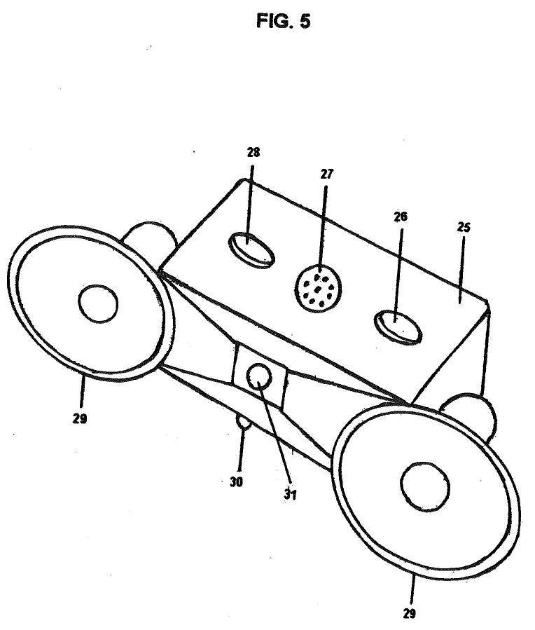

FIG. 6 are perspective views; the embodiments are, a GPS beacon, a glass window, an entry door, a deadbolt, a doorknob, a cover plate, a metal cutting saw.

### DETAILED DESCRIPTION OF THE PREFERRED EMBODIMENTS

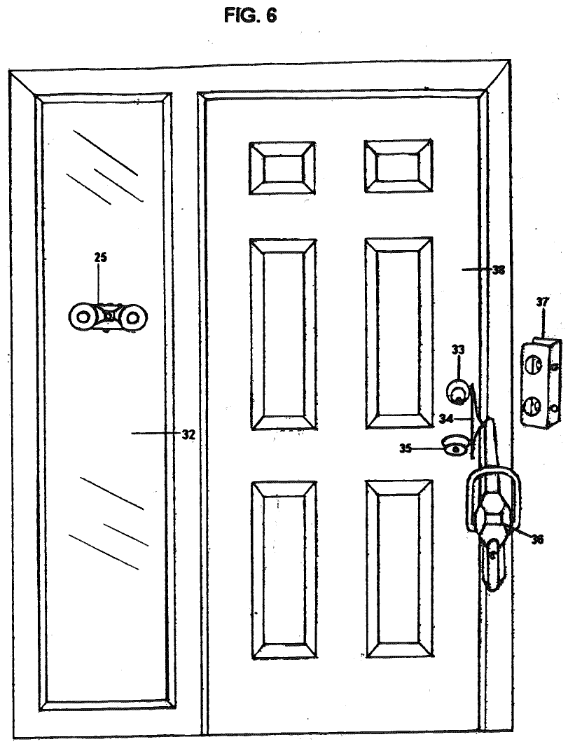

The Overdose Reviver Auto-Dial 911 Mayday Bracelet is described herein as “Mayday Bracelet” Wherein detecting an unconscious overdosed person's life-threatening low heart rate, wherein said Mayday Bracelet 6 is a life-saving method, wherein a piercing alarm beeps, a pre-recorded mayday message will automatically be dispatched to a 911 operator revealing nature of the emergency, personal information, location, GPS coordinates, requesting sending paramedics, wherein remotely activating a detached GPS beacon 25 with a high decibel siren 27.

Wherein said Maday Bracelet 6 is designed for a left wrist 9 or a right wrist 10, it must be positioned same as an upside-down wristwatch, wherein a display screen frame 5 and heartbeat sensors 1,2, are positioned on the underside of a wrist, wherein a Velcro clasp with a direction label 8 is positioned on the topside of a wrist.

Wherein a wristband 7 is constructed of nylon and said Velcro clasp, said Velcro clasp ensures a taut fit, whereas said heartbeat sensors 1,2, are snug against a radial artery. Said direction label 8 stitched on top of said Velcro clasp, “Top Left Top Right” indicates the correct rotation for either wrist, whereas said heartbeat sensors 1,2, will contact the left wrist radial artery 11 or the right wrist radial artery 12. Wherein said heartbeat sensors 1,2, and adjacent temperature sensor 3 relays data to a microprocessor chip 4. Wherein said temperature sensor data helps to prevent a false alarm. Wherein said heartbeat sensor 1,2, data detects an unconscious overdosed person's life-threatening low heart rate.

Wherein an alarm's open switch closes activating the programmed functions, wherein said GPS beacon 25 with a high luminous flashing bulb 31, a high decibel siren 27, wherein automatically dispatching said pre-recorded mayday message to a 911 operator. Wherein said mayday message is generated by pressing a record button 23, recite into a microphone 24 a suggested mayday message on a label 13 stitched inside said wristband 7. “Mayday I am unconscious due to an overdose my name is John Doe, my address is 123 Sample St. or I am homeless, the crossroads are Sample Rd. and Sample Dr. the GPS coordinates are longitude 21, latitude 19 please send paramedics. Wherein said alarm is activated, a piercing alarm beeps 24, remotely activates a detached GPS beacon 25, a siren speaker 27, and automatically dispatches a 911 mayday message.

Wherein button 17 is depressed, GPS coordinates illuminate on display screen 22, wherein said Mayday Bracelet 6 power on/off button is 20, wherein a button to manually call 911 is 16, wherein a microphone 14 to communicate with a 911 operator, wherein a battery charging inlet 15, wherein a LED light 18 on said display screen 22 flashes to a heartbeat rhythm.

Wherein said GPS beacon 25 features are; a power on button 26, a siren speaker 27, a power off button 28, an eyelet 30 for a string lanyard necklace, a high luminous flashing light bulb 31, suction cups 29. FIG. 6 illustrates a residence entry door 38, a street facing glass window 32, a deadbolt 33, a door knob 35, a deadbolt and door knob cover plate 37, a metal cutting saw 36, a 12″ metal cutting blade 34. Wherein said GPS beacon can be secured to a glass window via suction cups, or a homeless person can wear said GPS beacon as a necklace via said eyelet.

Said metal cutting saw with said metal cutting blade, demonstrates how a responder can gain entry through a locked door without busting said door by using a battering ram, wherein to slice into said door close to the door jam, cutting through said deadbolt and said doorknob shaft, easily prying said door open. Repairs to said door is covering over said door slice with said deadbolt doorknob cover plate.

What is claimed:

1. The Mayday Bracelet is a wrist wearable electronic automated heart monitoring device;
wherein heartbeat sensors detects an unconscious overdosed person's life-threatening low heart rate; an alarm emits a piercing beep; automatically dispatches a pre-recorded audio generated mayday message to a 911 operator revealing; nature of the emergency; personal information; location; GPS coordinates and requesting paramedics; remotely activates a detached GPS beacon with siren; a temperature sensor prevents a false alarm.

2. Any wrist wearable electronic automated heart monitoring device; or any similar miniature finger wearing heart monitoring device; whereby any means of detecting an unconscious person's life-threatening low heart rate; an alarm emits a piercing beep;
automatically dispatches a digitally generated computer program mayday message to a 911 operator revealing; nature of the emergency; personal information; location; GPS coordinates; and requesting paramedics.

3. Any wrist wearable electronic automated heart monitoring device; or any similar miniature finger wearing heart monitoring device; whereby any means of detecting an unconscious person's life-threatening low heart rate; an alarm emits a piercing beep;
automatically dispatches an artificial intelligence generated computer program mayday message to a 911 operator revealing; nature of the emergency; personal information;
location; GPS coordinates; and requesting paramedics.

4. Any wrist wearable electronic automated heart monitoring device; or any similar miniature finger wearing heart monitoring device; whereby any means of detecting an unconscious person's life-threatening low heart rate; an alarm emits a piercing beep;
automatically dispatches a cellular phone generated computer program mayday message to a 911 operator revealing; nature of the emergency; personal information;
location; GPS coordinates; and requesting paramedics.

5. Any wrist wearable electronic automated heart monitoring device; or any similar miniature finger wearing heart monitoring device; whereby any means of detecting an unconscious person's life-threatening low heart rate; automatically remotely activates a detached GPS beacon with a siren.
